(19) United States
(12) Patent Application Publication
(10) Pub. No.: US 2016/0128617 A1
(43) Pub. Date: May 12, 2016

(54) SOCIAL CUING BASED ON IN-CONTEXT OBSERVATION

(71) Applicant: Intel Corporation, Santa Clara, CA (US)

(72) Inventors: Margaret Morris, Portland, OR (US); Stanley Mo, Portland, OR (US); Carl S. Marshall, Portland, OR (US); Giuseppe Raffa, Portland, OR (US); Alexandra C. Zafiroglu, Portland, OR (US); Joshua Ratcliff, San Jose, CA (US)

(21) Appl. No.: 14/537,107

(22) Filed: Nov. 10, 2014

(51) Int. Cl. A61B 5/16; G09B 19/00

(52) U.S. Cl. CPC A61B 5/16; G09B 19/00

(57) ABSTRACT

System and techniques for social cuing based on in-context observation are described herein. A plurality of context attributes about a user and a subject may be received. A period in which an interpersonal interaction between the user and the subject occurs may be determined. A social-interaction-emotional-state may be determined during the period from the plurality of context attributes using a pre-defined classification methodology. A feedback response may be generated based on the determined social-interaction-emotional-state. The feedback may be presented to the user.

### TECHNICAL FIELD

Embodiments described herein generally relate state monitoring of biophysical indicia, and more specifically to social cuing based on in-context observation.

### BACKGROUND

Humans engage in interpersonal interactions that may include both verbal and non-verbal communication. Examples of non-verbal communication may include facial expressions, biophysical changes (e.g., becoming flushed, dilating pupils, etc.), body posturing, etc. Different people integrate verbal and non-verbal communications differently. These differences may be observed between groups (e.g., cultures, nations, gender, etc.) as well as between individuals within a group. Thus, two different people may arrive at different semantic conclusions to a conversation in which both are exposed to the same verbal and non-verbal material.

Non-verbal communication may indicate an underlying emotional response by a participant in a conversation. Classification systems to quantify this emotional response have been developed, such as the Facial Action Coding System (FACS), by Ekman & Friesen, or the Specific Affect Coding System (SPAFF), by Gottman & Krokoff.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a diagram of an example of an environment for social cuing based on in-context observation, according to an embodiment.

FIG. 2 illustrates a diagram of an example of an environment for social cuing based on in-context observation where the parties are remote from each other, according to an embodiment.

FIG. 3 illustrates a block diagram of an example of a system for social cuing based on in-context observation, according to an embodiment.

FIG. 4 illustrates a functional diagram of an example of a system for social cuing based on in-context observation, according to an embodiment.

FIG. 5 illustrates a functional diagram of an example of a node for social cuing based on in-context observation, according to an embodiment.

FIG. 6 illustrates a diagram of an example of a network of participants in an interpersonal interaction, according to an embodiment.

FIG. 7 illustrates a flow diagram of an example of a method for social cuing based on in-context observation, according to an embodiment.

FIG. 8 is a block diagram illustrating an example of a machine upon which one or more embodiments may be implemented.

### DETAILED DESCRIPTION

As noted above, interpersonal interactions often involve both verbal and non-verbal components. Also as noted above, different people may integrate (e.g., interpret) these components differently in an interaction. The reasons for the varied understanding of communication components such as non-verbal communication between two people may include upbringing (e.g., culture, life experience, etc.) and cognitive variations (e.g., autism, anxiety disorders, sociopathy, etc.), among others. The varied understanding of a conversation between two people may give rise to misunderstandings, or other sub-optimal results.

Traditionally, a person learns to alter unacceptable, or other sub-optimal, behavior in interpersonal interactions by observing one's own behavior and that of the other party to the interaction (e.g. reflection, reviewing video, each party taking notes, etc.) to assess the outcome of their own behavior in the interaction. For example, if a person begins discussing a surgery they observed over lunch, that person may observe disgust in other diners and realize that such a discussion may be inappropriate for this type of interpersonal interaction.

To facilitate interposal interactions (e.g., to reduce misunderstandings between the parties, or otherwise improve outcomes) a social cuing engine may monitor the parties of the interpersonal interaction, assess social cues during the interaction, and provide contextual feedback to a user. For example, the social cues of a party to the conversation (e.g., e.g., frowning, looking away, etc.) may be observed and interpreted (e.g., to interest, disinterest, anger, disgust, pleasure, displeasure, etc.) for the user, and a notification (e.g., text, tone, haptic signal, etc.) given to the user. In an example, the social cuing engine may use inputs from several sensors positioned so as to observe the interpersonal interaction. In an example, where the sensor is on another party's, device, an access system may provide access to the sensor information based on the context of the interpersonal interaction. By using the social cuing engine, a tireless and inexpensive real-time analysis may provide the user with mechanical ability to perceive non-verbal communications in others, perhaps beyond their own capabilities, and adjust their own behavior to achieve better outcomes in interpersonal interactions.

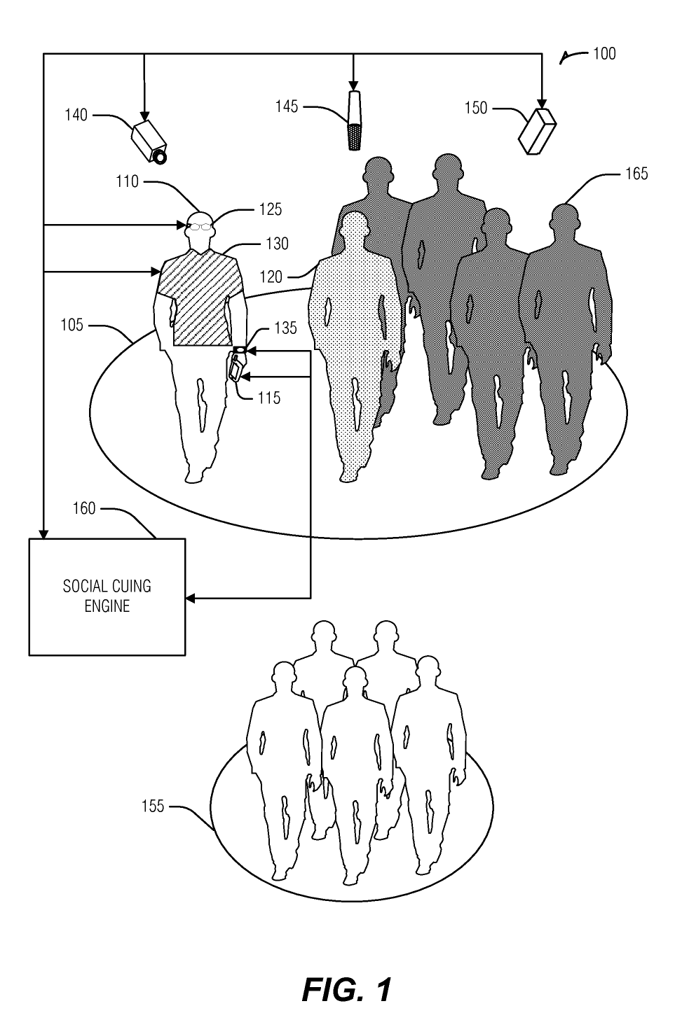

FIG. 1 is a diagram of an example of an environment 100 for social cuing based on in-context observation, according to an embodiment. The environment 100 may include a social cuing engine 160, an interpersonal interaction (e.g. conversation, gathering, party, interview, etc.) 105 including a user 110, a subject 120, and possibly other people 165. The social cuing engine 160 may determine that interpersonal interaction 105 has occurred between the user 110 and the subject 120; such as a conversation, meal, etc.; based on a plurality of context attributes collected by one or more attribute collectors.

The plurality of context attributes of the interpersonal interaction 105 may be collected by one or more attribute collectors in the environment 100 (e.g. on or in a structure, on the user 110, the subject 120, or the other people 165) positioned to observe the interpersonal interaction 105. Sensors on the attribute collectors may include any or all of cameras, microphones, positional sensors (e.g., gyroscope, accelerometers, positioning systems, proximity sensors, etc.), thermometers, chronographers, etc. Example attribute collectors may include a personal device 115 (e.g. a smartphone, etc.), a wearable device (e.g., smart watch 135, smart shirt 130, or smart glasess 125), an environmental camera 140 (either still or video camera), an environmental microphone 145, or other environmental devices 150 (e.g., a thermometer, proximity sensor, etc.). In an example, at least one attribute collector is operated by at least one of the user 110 or the subject 120.

Example context attributes that may be collected include facial expressions, vocal tone, heart rate, galvanic skin response, body temperature, or other biophysical indicia. In an example, the context attributes may also include environmental temperature, humidity, location, date (e.g., time, date, day-of-week, holidays, etc.), or other context attributes of the environment 100. In an example, the context attributes may be classified as an emotional context attribute (e.g. happy, angry, etc.), a physical context attribute (e.g. heart rate, body temperature, etc.), an interest context attribute (e.g. likes fishing, dislikes bowling, etc.), an identification context attribute (e.g. name, device id, etc.), a social status context attribute (e.g. PhD, CEO, etc.), or an environmental context attribute (e.g. temperature, humidity, etc.).

The one or more attribute collectors (115, 125, 130, or 135) may be communicatively coupled (e.g. via a wireless network) to the social cuing engine 160 to process the collected context attributes. As some of the context attributes may come from different individuals, those individuals may desire some control over the dissemination of the context attributes from their devices. Thus, in an example, the social cuing engine 160, or a user's device (e.g., mobile device 115), may include an access control mechanism to control access to a non-empty set of device-present context attributes (e.g. context attributes collected or stored on a device within the user's control). This set of device-present attributes is a subset of the plurality of context attributes. In an example, the social cuing engine 160, or the device upon which the context attribute is stored or collected, may implement an access control list (ACL) for these device-present attributes. In an example, the ACL is under control of the party controlling the device that either stored or collected the context attribute; this party is known as the provider. In an example, the subject 120 is the provider. Further, the ACL specifies eligible recipients to specific context attributes.

In an example, the ACL includes role-based access for a portion of the set of device-present attributes. Roles in the role-based access may correspond to positions (e.g., relationships between participants) in interpersonal communications. In an example, the roles are based on a physical relationship between parties in the interpersonal interaction 105. Examples of these roles may include a person that the provider came with (e.g., companion), a current party to the interaction (e.g., who I'm here with, a participant, etc.), whom I'm traveling with, etc. In an example, the positions in interpersonal communications include a social status of communicating persons (e.g. employer, employee, co-worker, relative, parent, child, spouse, education level, celebrity, popularity, authority, etc.). In an example, the social status of the user 110 and the subject 120 are determined from social media profiles of at least one of the user 110 or subject 120. In an example, the social status includes professional roles between the user 110 and the subject 120 (e.g. supervisor/subordinate, vendor/buyer, etc.).

With roles delineated by interpersonal interaction conventions, users may intuitively allow access to the context attributes by others in the interpersonal interactions. For example, attribute collectors may be on the user 110 and subject 120. Each of the user 110 or subject 120 may control access to the context attributes collected by the attribute collectors in their respective control. The social cuing engine 160 may determine they are co-workers based on one of the user's 110 or subject's 120 social network profiles. The subject 120 may have allowed co-workers access to physical and environmental context attributes collected from her attribute collectors via the ACL. Thus, the user 110, determined to be a co-worker of the subject 120, may have access to the physical and environmental context attributes collected from the subject's 120 attribute collectors. These context attributes may be added to the plurality of context attributes processed by the social cuing engine 160 for the user 110.

The social cuing engine 160 may analyze collected attributes against a pre-determined classification methodology to determine a social-interaction-emotional-state for the interpersonal interaction 105. As used herein, the pre-determined classification methodology is a classification mechanism of the interpersonal interaction 105 that applies human emotional modeling and uses a portion of the context attributes as inputs. Examples of such classification mechanisms include SPAFF, FACS (and FACS derivatives such as EMFACS), and the like. However, any mechanism that may accept context attributes and produce an emotional state of one or more participants to the interpersonal interaction 105 may be the pre-determined classification methodology. Social-interaction-emotional states may be either an emotional state of the interaction (e.g., tense, confrontational, jovial, casual, etc.) or of individual participants (e.g., anger, disgust, joy, happiness, etc.).

The social cuing engine 160 may use the social-interaction-emotional-state of the interpersonal interaction 105 to generate a feedback response and present it to the user 110. In an example, the feedback response includes a visual component. In an example, the visual component is at least one of a text message, an instant message, a video, or an icon. For example, the user 110 may be presented with text message via the personal device 115 suggesting that the user 110 change the topic when the social-interaction-emotional-state (e.g., of the subject 120 or other people 165) is uncomfortable, for example, because the user 110 told an inappropriate anecdote. In an example, the visual cue may include a change in the color of an audience member's clothing, akin to a mood ring. Such a color change may signify an emotional change informing the speaker of the audience member's reception of the speaker's material. In an example, a representation of the audience member may change color, in a fashion similar to changing the color of the audience member's clothes, where the audience member is not personally present, such as on a telephone call. Such feedback to the speaker may often be absent in remote interpersonal communications, such as conference calls.

In an example, the feedback response includes a haptic (e.g. touch-based, etc.) component. For example, the feedback response may include a gentle squeezing of the user's 110 wrist via the smart watch 135. This squeezing may correspond with calming a person, and so this feedback response may be used when the social-interaction-emotional-state the user 110 is upset (e.g., angry). In an example, the feedback response includes an audio component. Examples of audio components may include soothing music, rhythmic beeping, or other sounds designed to invoke an emotional response in the subject 110 to correct a perceived problem of the interpersonal interaction 105, such as a short temper, waning attention, etc.

In an example, the feedback response includes an environmental component. Example environmental components may include temperature or humidity adjustments, for example, via the smart shirt 130, or other capable garment. In an example, the environmental feedback response may include environmental controls available in the venue of the interpersonal interaction 105. Other environmental components may include ambient lighting, or other lighting, ambient sound, furniture adjustments (e.g., making a controllable chair less comfortable to sharpen attention), etc.

The feedback response is configured to change the subject's 110 behavior to provide a better outcome for the interpersonal interaction 105. In an example, the social cuing engine 160 may modify a subsequent feedback response based on a deviation of an interaction result for the user 110 from a desired interaction result following receipt of the feedback response by the user. Thus, the social cuing engine 160 adapts feedback responses as the results from previous responses becomes known. For example, the user 110 may be presented with a feedback response notifying the user 110 to change the topic of discussion when the social-interaction-emotional-state changes from happy to angry, and the user 110 complies. However, after the first feedback response, the social-interaction-emotional-state remains angry, indicating that the first feedback response was at least partially ineffective. For example, the subject 120 may actually be angry because of the user's 110 vocal tone, and not necessarily the topic of conversation. The social cuing engine 160 may present a subsequent feedback response to the user 110, for example, indicating that the user 110 should change their vocal tone. This process may be repeated as necessary to facilitate the interpersonal interaction 105 on behalf of the user 110.

In an example, there may be a second interpersonal interaction 155 in which the user 110 is not currently participating. The second interpersonal interaction 155 may, for example, be another conversation across the room, or it may be a gathering at a venue to which the user 110 has not yet arrived. When encountering the second interpersonal interaction, the social cuing engine 160 may provide a recommendation as to whether the second interpersonal interaction 155 would be a good fit for the user 110. This recommendation may be based both on the social dynamics of the second interpersonal interaction 155 (e.g., context attributes) or on a commonality of interests between participants of the second interpersonal interaction 155 and the user 110.

In an example, the social cuing engine 160 may receive observations of the second interpersonal interaction 155 including attributes of parties to the other interpersonal interaction 155 and a social-interaction-emotional-state of the other interpersonal interaction 155. The attributes may include another plurality of context attributes (e.g., pertaining to the second interpersonal interaction 155 and not necessarily the first inter personal interaction 105) that may also include a set of interest attributes. The social cuing engine 160 may assess the suitability of the other interpersonal interaction 155 for the user based on both the social-interaction-emotional-state of the other interpersonal interaction 155 and the set of interest attributes. The social cuing engine 160 may also provide the user 110 with a recommendation as to whether to join the other interpersonal interaction 155 based on the assessed suitability of the other interpersonal interaction 155. In an example, the recommendation includes directions to bring the user 110 to the other interpersonal interaction 155. For example, the user 110 may walk into a bar wearing smart glasess 125 with a heads-up display and a camera. The camera and data from participants to the second interpersonal interaction 155 may gather the context attributes of those participants. Further, the social cuing engine 160 may access interest data of the participants, such as that found in social networking applications. The social cuing engine 160 may compile the interest data to determine a compatibility between the user 110 and the participants. Further, the social cuing engine 160 may assess the tenor of the conversation using the techniques described above. Thus, if the conversation appears to be going well, and there is at least a partial match in interests, the social cuing engine 160 may direct an arrow to be placed on the heads-up display of the glasess 125 pointing in the direction of the second interpersonal interaction 155. Other example directions may include a description of clothing for the participants, a participant's name, nick name, a room number, etc.

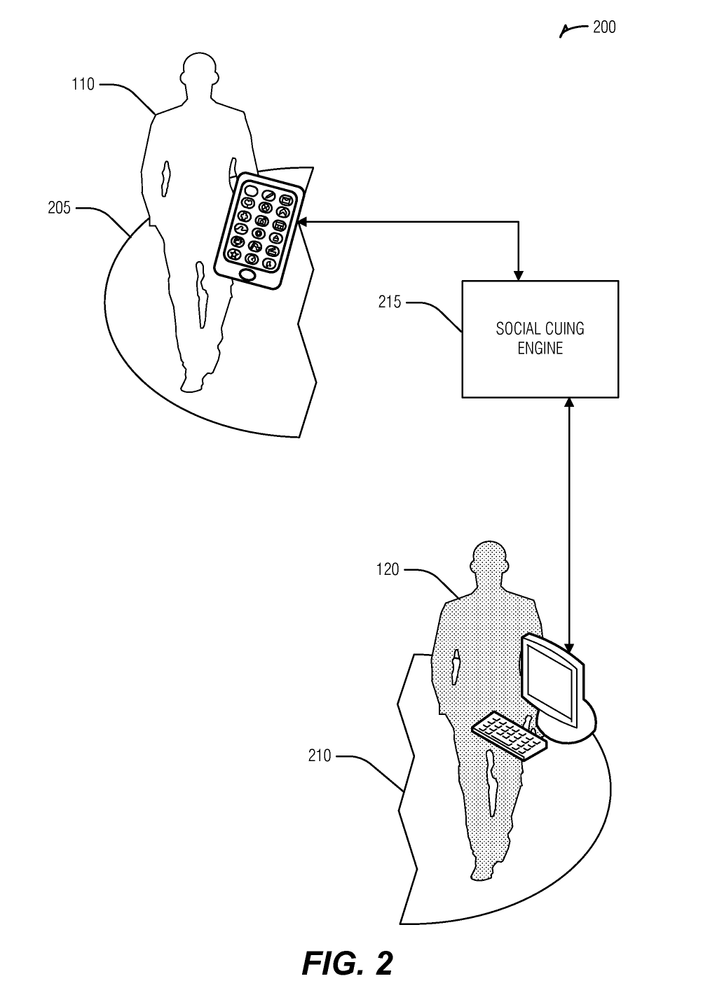

FIG. 2 illustrates a diagram of an example of an environment 200 for social cuing based on in-context observation where the parties are remote from each other, according to an embodiment. The environment 200 is an alternative configuration of environment 100 as described above with respect to FIG. 1, whereby the user 110 and the subject 120 are remote from each other, in a first location 205 and a second location 210 respectively. The social cuing engine 215 works as the social cuing engine 160 described above, with the additional capabilities to facilitate the interpersonal interaction encompassing the first location 205 and the second location 210. That is, the first location 205 and second location 210 are collectively similar to the interpersonal interaction 105 described above with respect to FIG. 1. As also described above, the first location 205 and the second location 210 may be configured to contain a plurality of attribute collectors.

The user 110 and subject 120 may communicate over a network, such as via telephone, video link, text chat, etc. The plurality of attribute collectors collect the context attributes of the user 110 and the subject 120 from both of the first location 205 and the second location 210. The plurality of context attributes may be analyzed by the social cuing engine 215, for example, as described above with respect to the social cuing engine 160. The social cuing engine 215 may determine that an interpersonal interaction is occurring between the user 110 and the subject 120. The social cuing engine 215 may determine, from the context attributes and a pre-determined classification methodology, a social-interaction-emotional-state of the interpersonal interaction. The social cuing engine 215 may then generate and present a feedback response to the user based on the social-interaction-emotional-state.

An example scenario involving the user 110 and the subject 120 being remote from each other may include the user 110 in first location 205 and the subject 120 in second location 210 having a video conversation about music. The social cuing engine 215, may determine that the social-interaction-action-emotional-state of the interpersonal interaction is happy. The user 110 may begin to get upset and gesticulate wildly when discussing a musician the user 110 doesn't like. The subject 120 may respond nonverbally to the wild gestures of the user 110 so as to indicate that the subject 120 is uncomfortable. The social cuing engine 215 may determine that the social-interaction-emotional-state for the subject 120 indicates this uncomfortableness and present the user 110 a feedback response notifying the user to calm down in order to improve the outcome of the interpersonal interaction.

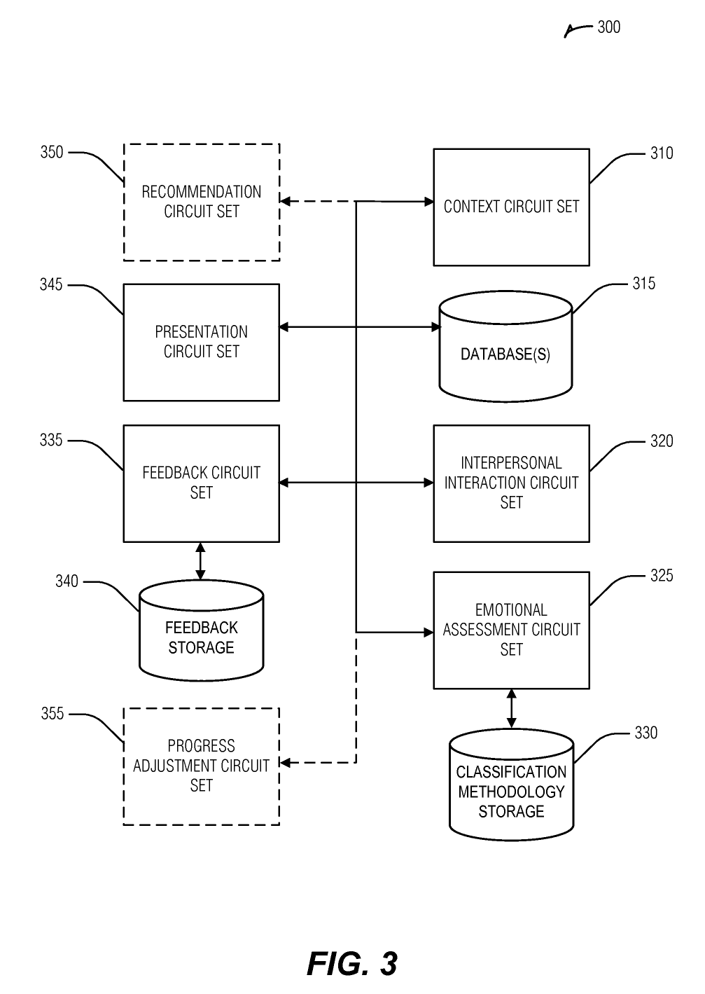

FIG. 3 illustrates a block diagram of an example of a system 300 for social cuing based on in-context observation, according to an embodiment. The system 300 may implement the social cuing engines 160 and 215 described above. The system 300 may include several communicatively coupled components when in operation, including a context circuit-set 310, databases 315, an interpersonal interaction circuit-set 320, an emotional assessment circuit-set 325, a classification methodology storage 330 (e.g., a database), a feedback circuit-set 335, a feedback storage 340 (e.g., a database), and a presentation circuit-set 345. One or all of the circuit-sets in system 300 may reside on disparate hardware platforms (e.g. different physical nodes, virtual nodes, different operating systems, etc.).

The context circuit-set 310 may be configured to receive a plurality of context attributes about a user and a subject (e.g. from sensors embedded in a wearable device or smartphone, from sensors in the environment, etc.). The interpersonal interaction circuit-set 320 may be configured to determine a period in which an interpersonal interaction between the user and the subject occurs (e.g. the user and subject are having a conversation, etc.). Determination of the period may be based on identifying the voices of both the user and the subject when close (e.g., within conversational distance or earshot) to each other. Other context attributes may be used, such as an event (e.g., party, meeting, etc.) that both are attending, proximity of the participants, etc.

In an example, a plurality of context attributes include at least one of an environmental attribute (e.g. temperature, humidity, etc.), an emotional attribute (e.g. happy, sad, angry, etc.), a physical attribute (e.g. heart rate, body temperature, facial expression, etc.), an interest attribute (e.g. literature, current events, sports, food, etc.), and identification attribute (e.g. name, device id, etc.), or a social status attribute (e.g. profession, education, circle of friends, etc.). In an example, the plurality of context attributes are derived from a sensor of a device (e.g. smartphone, wearable, etc.), the sensor positioned so as to observe a physical area of the interpersonal interaction (e.g. on a user or subject, monitoring the area including the user and subject, etc.). In an example, the device is operated by at least one of the user or the subject. For example, a user and subject may be conversing in a café. The user and subject may be using a combination of devices (e.g. smartphones, wearables, etc.). The sensors embedded in the devices may collect the temperature and humidity of the café, the heart rates of the user and subject, the physical position of the user with respect to the subject. The café may also have sensor devices such as a camera or a microphone that monitor the physical space that the user and subject occupy. In an example, the context attributes of the interpersonal interaction may be members of the plurality of context attributes to further refine the environment in which the interpersonal interaction is taking place.

In an example, an access control mechanism may control access to a set of device-present (e.g. on a smartphone, wearable, etc.) context attributes. In this example, the set of device-present attributes may be a subset of the plurality of context attributes. The access control mechanism may use an ACL controlled by a provider who controls the device. The ACL may also include identification (e.g. user name, device id, etc.) of a potential recipient of the set of on-device attributes. In an example, the subject is the provider. In an example, the ACL includes role-based access for a portion of the set of on-device context attributes. Roles in the role-based access may correspond to positions (e.g. relationships between participants such as people one is in a conversation with, traveling with, etc.) in interpersonal communications. In an example, the positions in interpersonal communications include a social status (e.g. education, circle of friends, etc.) of communicating persons. In an example, the social status of the user and the subject are determined from social media profiles (e.g. personal and professional social networking websites, etc.) of at least one of the user or subject. In an example, the social status includes professional roles between the user and the subject (e.g. boss, subordinate, co-worker, sibling, etc.).

Returning to the café example, the device of the subject or user contains context attributes collected from sensors on the device, such as microphones or cameras of the participant's smart phones. The subject, as a provider of these on-device context attributes, defines shared and unshared attributes (e.g., creates the ACL) via a user interface on the device. The user, as a recipient of the context attributes, restricted to the on-device context attributes permitted to the user, or a group to which the user belongs, based on the ACL. Likewise, the user, as provider, may define attributes as shared and not shared. Roles may be automatically assigned to the participants based on the position of the user and subject in the interpersonal interaction. Thus, the user and the subject may each be given the roles of “in conversation with” with respect to the other when the system interpersonal interaction circuit-set 320 determines that the subject is in a conversation with the user. Other roles may be determined via social networks of either the user or the subject. For example, the user may share interest, physical, and environmental context attributes with co-workers. If the subject is designated a co-worker, the subject will receive access the user's interest, physical, and environmental attributes.

The emotional assessment circuit-set 325 may be configured to determine a social-interaction-emotional-state, during the interpersonal interaction period, from the plurality of the context attributes using a pre-defined classification methodology. In an example, to determine a social-interaction-emotional-state, at least one of the plurality of context attributes is compared to a normalized value corresponding to the at least one of the plurality of context attributes to determine a variance. The variance may be used to determine the social-interaction-emotional-state from the pre-defined classification methodology. Thus, a norm for the user is tested against the determined social-interaction-emotional-state. The norm may be obtained via training (e.g., observing the user), set (e.g., via a user interface), or preconfigured (e.g., a baseline human norm may be used). When the social-interaction-emotional-state deviates from that norm, the deviation is the variance. In an example, the emotional assessment circuit-set 325 may be configured to retrieve, or otherwise access, the pre-defined classification methodology from a data storage mechanism, such as the database for classification methodology storage 330. Various classification methodologies may be stored in the database 330 and indexed to multiple interpersonal interactions and may be accessed by the emotional assessment circuit-set 325 when the system 300 is in use. For example, the SPAFF methodology may be indexed to interpersonal interactions between married couples or dating couples.

Returning to the café example, after its been determined that the user and the subject are engaged in an interpersonal interaction and context attributes corresponding to the user and subject have been collected (e.g. heart rate, images, audio, etc.), a social-interaction-emotional-state is determined using the pre-defined classification methodology and the context attributes of the user and subject. For example, the user told a joke and the subject frowns and the tone of the subject's voice and heart rate have changed. The context attributes in the period surrounding the joke are analyzed and it is determined that the subject was put-off by the joke when the classification methodology is applied to the nonverbal changes in the subject. It could be that the subject has had a bad day and the frown, etc. are unrelated to the joke. In an effort to provide more accurate results, the subject's reactions throughout the day may be collected to create a normalized emotional value for the day, or other time period. The normalized value may then be compared to the value related to the joke response to see if there is a variance. If there is no variance then the subject may not be put-off but the joke itself. However, if there is a variance, there is opportunity for the feedback response to provide a better outcome for the interpersonal interaction (e.g., to stop telling that joke to the subject).

The feedback circuit-set 335 may be configured to generate the feedback response based on the determined social-interaction-emotional-state. In an example, the feedback response includes a haptic component. In an example, the feedback response includes a visual component. In an example, the visual component is at least one of a text message, an instant message, a video, or an icon. In an example, the feedback response includes an audio component. In an example, the feedback response includes an environmental component. The feedback circuit-set 335 may retrieve indexed feedback responses, or feedback response templates, from a data store, such as feedback storage 340. Various feedback responses may be stored and indexed against multiple social-interaction-emotional-states and may be accessed by the feedback circuit-set 335 when the system 300 is in use. For example, a feedback response indicating a change in topic could be indexed to a social-interaction-emotional-state of angry or bored. In an example, the feedback response may also be indexed by context attributes such as social status. Thus, in an interpersonal interaction between an employee and employer, the feedback response for a tense conversation may direct the employee user to calm down and diffuse the controversy more than a feed-back response for the same situation where the user and subject are peers. For example, in a classroom setting, the teacher can be alerted to changes in student states, such as inattention (e.g., “day-dreaming”), over-excitement, anxiety, etc. during a lesson or study period. Such feedback, e.g., in large classroom settings, may help teachers tune their instruction method to increase student engagement and thus positive outcomes.

The presentation circuit-set 345 may be configured to present the feedback response to the user. Thus, the presentation circuit-set 345 may include communication capabilities to, for example, send a text to the user's device. In an example, the presentation circuit-set 345 may control one or more components on the user's device, such as speakers, lights, the display, etc.

Returning again to the café example, the user has told a joke and the subject is determined to be offended by that joke. A feedback response corresponding to the subject being offended by the joke is selected for presentation to the user. The feedback response may include a haptic component, such as gently constricting a bracelet worn by the user, which may be understood by the user as an indication that someone was offended. The user could also have a device (e.g. smartphone) that renders (e.g., displays, performs, etc.) audio or visual components of the feedback response. Thus, the user may receive an icon (e.g. offended emoticon), a text message, instant message, or video (e.g. an animated representation of a person that is offended) letting the user know the subject is offended.

In an example, the user may be uncomfortable in the situation and is telling bad jokes as a coping mechanism. Thus, making the user more comfortable may provide a better interpersonal interaction outcome. In this example, the feedback response may be directed to the user's environment instead of being directed at to the user. For example, the user may wear clothing capable of cooling the user, providing a deodorizing effect, wicking sweet, etc. The feedback response may instruct the clothing to cool the user in an effort to make the user more comfortable. In an example, the clothing may also be configured with a mechanism to provide a comforting brush of the user's arm, or simulate a hug, or other comforting actions. With the user more comfortable, a better interpersonal interaction may be achieved.

In an example, the system 300 may optionally include a recommendation circuit-set 350. The recommendation circuit-set 350 may provide recommendations to the user as to which interpersonal interactions to join, such as which conversations in a party are likely to please the user. Thus, the recommendation circuit-set 350 may be configured to receive observations of a second interpersonal interaction including attributes of parties to the second interpersonal interaction and a second social-interaction-emotional-state for the second interpersonal interaction. These attributes may include a second plurality of context attributes including a set of interest attributes. The recommendation circuit-set 350 may be configured to asess suitability of the second interpersonal interaction for the user based on both the second social-interaction-emotional-state and the set of interest attributes. The recommendation circuit-set 350 may also be configured to provide the user with a recommendation as to whether to join the second interpersonal interaction based on the assessed suitability of the second interpersonal interaction. In an example, the recommendation includes directions to bring the user to the second interpersonal interaction.

An example, of the recommendation circuit-set 350 in action may include the user participating in a conversation with the subject at party. The user and subject finish their conversation and the user would like to engage in another conversation. There may be several groups of people having conversations in the room. Each of the groups' attributes may be collected and analyzed by the recommendation circuit-set 350 to determine the social-interaction-emotional-state and interests of each group. The user's interests may be compared to those of each group to determine suitable groups for the user to join. Further, the social-interaction-emotional-states of the groups may be ordered to asess which groups are engaged in better interpersonal interactions. Once the determination is made, the user may receive a feedback response directing the user to one of the suitable groups. For example, the feedback response may include on descriptions or images of members of the selected group.

In an example, the system 300 may optionally include a progress adjustment circuit-set 355. As previously described, the feedback response is intended to improve interpersonal interaction outcomes for the user. The progress adjustment circuit-set 355 facilitates incremental improvements over the course of several feedback responses during an interpersonal interaction. Thus, the progress adjustment circuit-set 355 may be configured to modify a subsequent feedback response based on a deviation of an interaction result for the user from a desired interaction result following receipt of the feedback response by the user.

Returning to the café example, the user may receive a feedback response notifying the user that the subject was offended by the joke and suggesting that the user change the topic. The user may then tell another joke on a different subject. The subject may be offended by the second joke. The user may receive a subsequent feedback response telling the user that subject took offense again and suggesting that the user make a change (unspecified by the feedback response) other than a topic change, as changing the topic did not appear to improve the interpersonal interaction outcome. Additionally, the interaction may be stored and the subject may be identified as a person who does not like most jokes. Thus future feedback responses to the user pertaining to this subject may include instructions to avoid jokes.

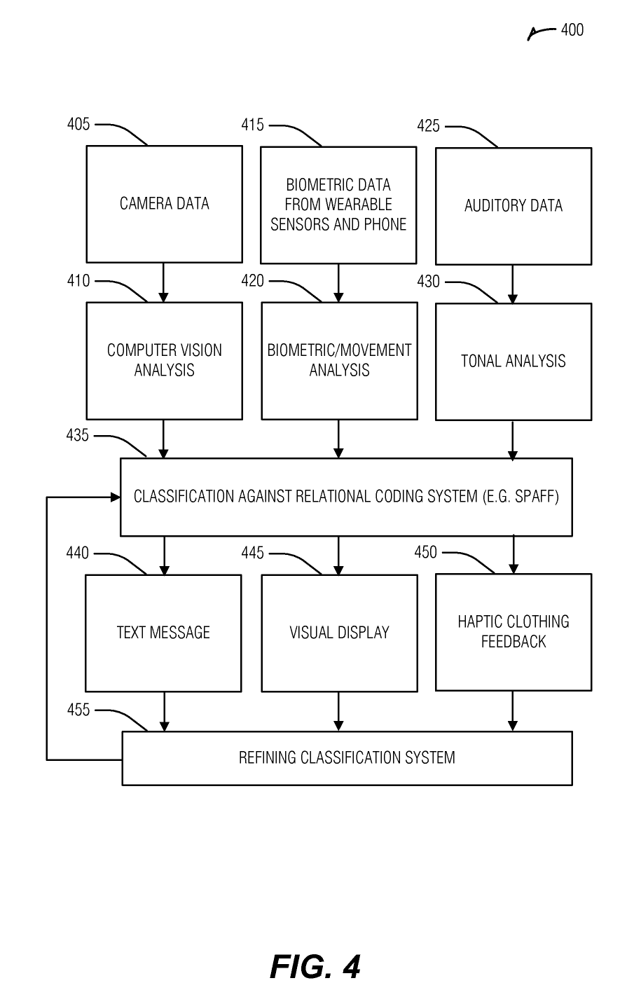

FIG. 4 illustrates a functional diagram of an example of system 400 for social cuing based on in-context observation, according to an embodiment. The system 400 is an example of the system 300 described in the description of FIG. 3. The system 400 may include context attributes including camera data 405, biometric data from wearable sensors and phone 415, and auditory data 425. The context attributes may be respectively analyzed using computer vision analysis 410 (e.g., to detect physical movements of the participants, such as facial movements underlying facial coding mechanisms), biometric/movement analysis 420, or tonal analysis 430. The system 400 may also include a classification against relational coding system 435 to, for example, provide the social-interaction-emotional-state. The system 400 may provide feedback response in the form of a text message 440, visual display 445, or haptic clothing feedback 450. The system 400 may also incorporate corrections, either via observed deviance from a desired result or entered by the user, via a classification system refinement mechanism 455, which may adjust the coding system 435.

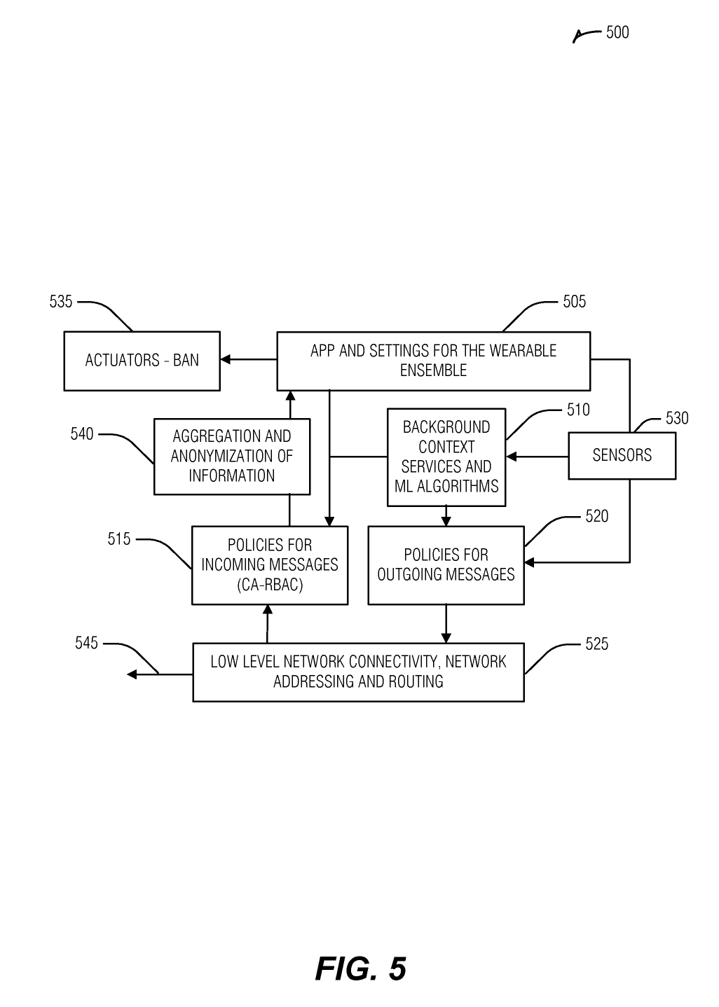

FIG. 5 illustrates a functional diagram of an example node 500 for social cuing based on in-context observation, according to an embodiment. The node 500 may include applications or settings for the wearable ensemble 505, policies for incoming messages 515, policies for outgoing messages 520, sensors 530 (e.g., to collect context attributes as described above), aggregation and anonymization of information 540, background context services and machine learning algorithms 510, and actuators (or other output mechanisms) in a body area network (BAN) 535 (e.g., to present feedback to the user as described above). The BAN allows the wearables on the user to be communicatively coupled to the node 500. The node 500 may also include network connectivity 525 to communicate with other devices 545 (e.g. smartphones, wearables, PC, etc.).

An example, use of the node 500 in the context of a social cuing engine (e.g., social cuing engines 160 or 215 described above), or the systems (e.g., system 300 or 400 as described above) may proceed as follows. The application 505 may provide an interface allowing the user to set incoming message policies 515 and outgoing message policies 520. For example, for the outgoing messages, the user may define what information may be shared with others, and, in an example, specifically what information may be shared with whom.

The exact identity of recipients may not be known in advance to the user. Thus, a Context-Aware RBAC (CA-RBAC) model may be provided to the user to manager with whom data may be shared. CA-RBAC differs from traditional RBAC models in that, instead of typical “roles” (administrator, user, etc.), a set of context—aware roles such as “came with me”, “my friend”, “near me”, “in a conversation with me”, etc. are defined. These context-aware roles may be stored in an application specific taxonomy. In this way the user may customize the recipients of her messages. Similar to the outgoing message policies 520, the user may setup CA-RBAC policies for incoming messages 515, in order to filter messages coming from a “relevant” set of users (e.g., people near the user, or in a conversation with him).

The network connectivity 525 receives incoming messages, delivers outgoing messages, keeps a dynamic routing table depending on the actual state of the wider social network (WSN) of people in the social situation (assuming we have one ID for each person in the WSN), and routing messages that still need to reach other nodes.

In an example, incoming messages may be anonymized or aggregated by component 540, for example, based on the policies set by the sender or receiver. The application 505 may provide output (e.g., feedback response) via the BAN 535 (e.g., via actuators, displays, etc.), based on the anonymized data. Further, the sensors 530 may continuously collect on-device context attributes (e.g., on the environment or on the person) that are then processed at component 510. The component 510 may employ background context, services, machine learning, or other facilities to infer changes in the inter person interaction (e.g., in user or group stress levels) or personal information and events. This data may be communicated to other interested parties according to the CA-RBAC model. The sensors 530 may also be integrated into the BAN 535.

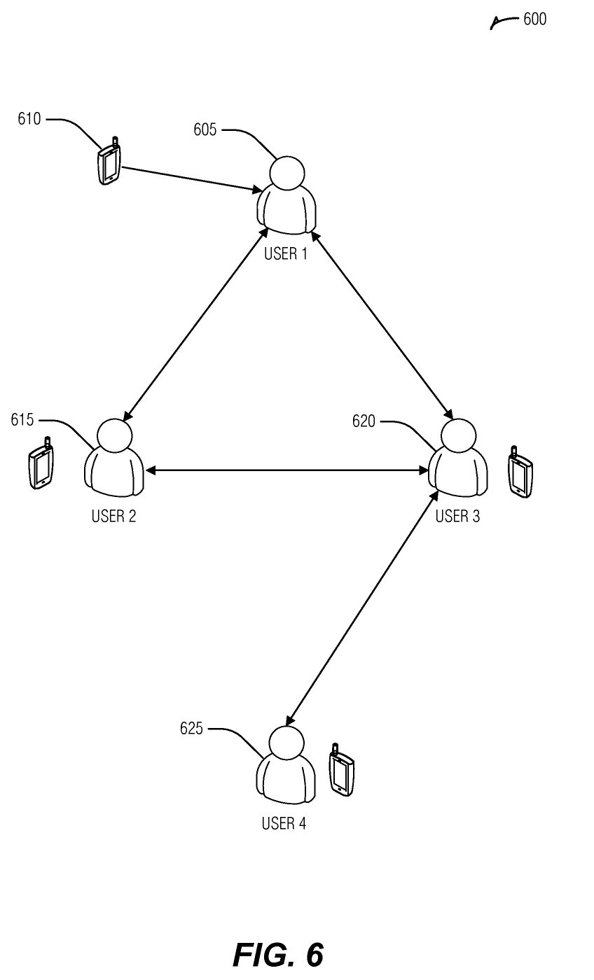

FIG. 6 illustrates a diagram of an example of a network 600 of participants in an interpersonal interaction. The network 600 may include a first user 605 and a device 610 with which the first user 605 may define sharable data similar to the access control mechanisms described above. The device 610 may be configured to have a user interface that allows the first user 605 to input attributes such as interests and configure the access control list for attributes collected from the device. The first user 605 may wear one or more wearable devices (e.g. glasess, clothing, wristband, etc.) that are communicatively coupled to the network 600. Sharable information may be downloaded to the wearable devices based on an ACL configured on the device 610. The network 600 may include a second user 615 and a third user 620 that have sharable information in devices connected to the network 600. The second user 615 and the third user 620 may be in an interpersonal interaction. The first user 605 may receive a feedback response on device 610 directing him to the suitable interpersonal interaction between the second user 615 and the third user 620. Upon joining the interpersonal interaction, the third user 620 may engage in a conversation with a fourth user 625. The sensors monitoring the third user 620 collect attributes that may be compared to normalized values for analysis to judge the third user 620's social-interaction-emotional-state. Upon classifying the social-interaction-emotional-state using a pre-defined classification methodology, the fourth user 625 may be presented a feedback response to assist in directing the conversation to a successful result.

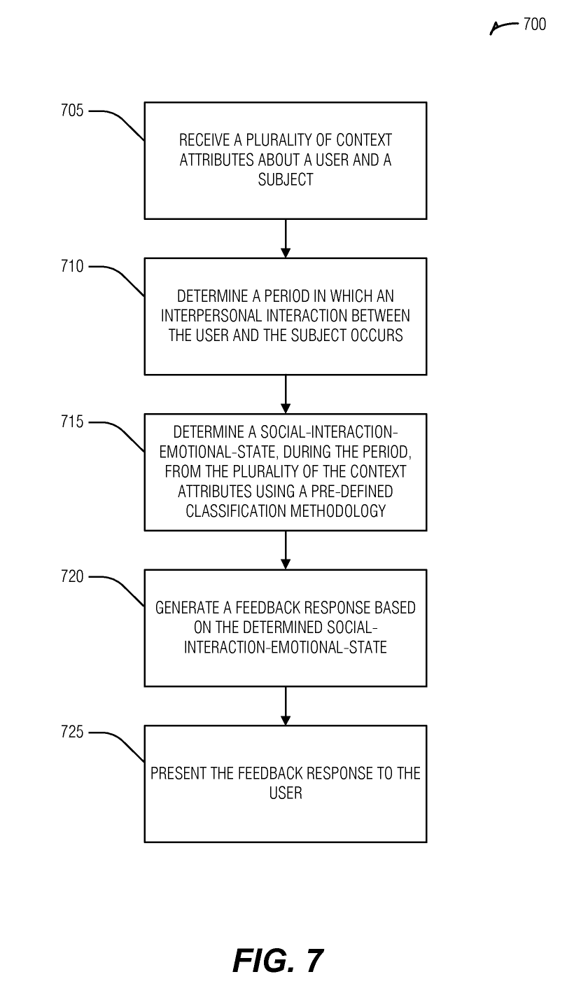

FIG. 7 illustrates a flow diagram of an example of a method 700 for social cuing based on in-context observation, according to an embodiment.

At operation 705, a plurality of context attributes may be received about a user and a subject. In an example, the plurality of context attributes includes at least one of an environmental attribute, an emotional attribute, a physical attribute, an interest attribute, an identification attribute, or a social status attribute. In an example, the plurality of context attributes are derived from sensor data from a sensor of a device where the sensor positioned so as to observe a physical area of the interpersonal interaction. In an example, the device is operated by at least one of the user or the subject.

In an example, the method 700 may optionally comprise an operation to control access to a set of at least one device-present context attribute, the set being a subset of the plurality of context attributes, based on an access control list controlled by a provider and identification of a potential recipient, the set having been granted access to the user as the potential recipient. In an example, the subject is the provider. In an example, the access control list includes role-based access for a portion of the set, roles in the role-based access corresponding to positions in interpersonal communications. In an example, the positions in interpersonal communications include a social status of communicating persons. In an example, the social status of the user and the subject are determined from the social media profiles of at least one of the user or subject.

At operation 710, the computer system determines a period in which an interpersonal interaction between the user and the subject occurs. As described above, this determination may be based on a number of factors. In an example, physical proximity between the user and the subject given a communications mechanism (e.g., voice) is a factor. In an example, the factor is an electronic communication in which both are parties (e.g., a video conference in which both the user and the subject are attendees).

At operation 715, the computer system may determine a social-interaction-emotional-state, during the period, from the plurality of the context attributes using a pre-defined classification methodology. In an example, to determine a social-interaction-emotional-state, at least one of the plurality of context attributes is compared to a normalized value corresponding to the at least one of the plurality of context attributes to determine a variance, the variance used to determine the social-interaction-emotional-state from the pre-defined classification methodology.

At operation 720, the computer system may generate a feedback response based on the determined social-interaction-emotional-state. In an example, the feedback response includes a haptic component. In an example, the feedback response includes a visual component. In an example, the visual component is at least one of a text message, an instant message, a video, or an icon. In an example, the feedback response includes an audio component. In an example, the feedback response includes an environmental component. At operation 725, the computer system presents the feedback response to the user.

In an example, the method 700 may optionally comprise receiving observations of a second interpersonal interaction including attributes of parties to the second interpersonal interaction and a second social-interaction-emotional-state. The attributes may include a second plurality of context attributes including a set of interest attributes. The method 700 may also optionally comprise assessing suitability of the second interpersonal interaction for the user based on both the second social-interaction-emotional-state and the set of interest attributes. The method 700 may also optionally comprise providing the user with a recommendation as to whether to join the second interpersonal interaction based on the assessed suitability of the second interpersonal interaction. In an example, the recommendation includes directions to bring the user to the second interpersonal interaction.

In an example, the method 700 may optionally comprise modifying a subsequent feedback response based on a deviation of an interaction result for the user from a desired interaction result following receipt of the feedback response by the user. In this example, the feedback response and the subsequent feedback response configured to modify a behavior of the user to achieve the desired interaction result for the interpersonal interaction.

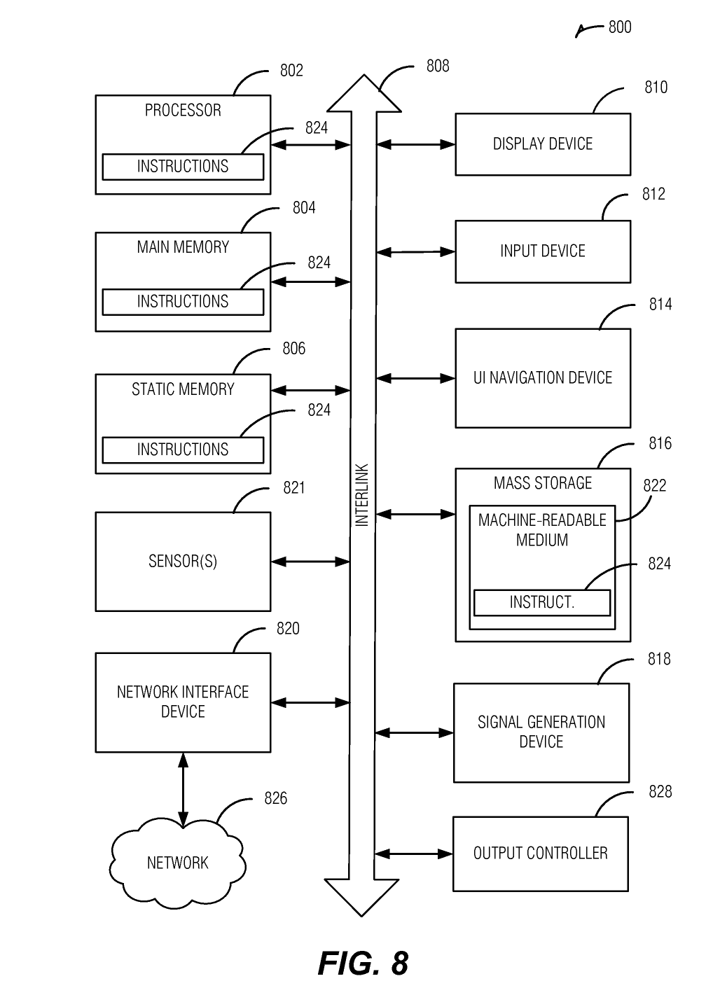

FIG. 8 illustrates a block diagram of an example machine 800 upon which any one or more of the techniques (e.g., methodologies) discussed herein may perform. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit-sets are a collection of circuits implemented in tangible entities that include hardware (e.g., simple circuits, gates, logic, etc.). Circuit-set membership may be flexible over time and underlying hardware variability. Circuit-sets include members that may, alone or in combination, perform specified operations when operating. In an example, hardware of the circuit-set may be immutably designed to carry out a specific operation (e.g., hardwired). In an example, the hardware of the circuit-set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit-set in hardware via the variable connections to carry out portions of the specific operation when in operation. Accordingly, the computer readable medium is communicatively coupled to the other components of the circuit-set member when the device is operating. In an example, any of the physical components may be used in more than one member of more than one circuit-set. For example, under operation, execution units may be used in a first circuit of a first circuit-set at one point in time and reused by a second circuit in the first circuit-set, or by a third circuit in a second circuit-set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

### ADDITIONAL NOTES & EXAMPLES

Example 1 may include subject matter (such as a device, apparatus, or system for social cuing based on in-context observation) comprising, a context circuit-set to receive a plurality of context attributes about a user and a subject, an interpersonal interaction circuit-set to determine a period in which an interpersonal interaction between the user and the subject occurs, an emotional assessment circuit-set to determine a social-interaction-emotional-state—during the period—from the plurality of context attributes using a pre-defined classification methodology, a feedback circuit-set to generate a feedback response based on the determined social-interaction-emotional-state, and a presentation circuit-set to present the feedback response to the user.

In Example 2, the subject matter of Example 1 may optionally include, wherein the plurality of context attributes includes at least one of an environmental attribute, an emotional attribute, a physical attribute, an interest attribute, an identification attribute, or a social status attribute.

In Example 3, the subject matter of any of Examples 1-2 may optionally include, wherein the plurality of context attributes are derived from sensor data from a sensor of a device, the sensor positioned so as to observe a physical area of the interpersonal interaction.

In Example 4, the subject matter of Example 3 may optionally include, wherein the device is operated by at least one of the user or the subject.

In Example 5, the subject matter of any of Examples 1-4 may optionally include an access control mechanism to control access to a set of at least one device-present context attribute, the set being a subset of the plurality of context attributes, based on an access control list controlled by a provider and identification of a potential recipient, the set having been granted access to the user as the potential recipient.

In Example 6, the subject matter of Example 5 may optionally include, wherein the subject is the provider.

In Example 7, the subject matter of any of Examples 5-6 may optionally include, wherein the access control list includes role based access for a portion of the set, roles in the role based access corresponding to positions in interpersonal communications.

In Example 8, the subject matter of Example 7 may optionally include, wherein the positions in interpersonal communications include a social status of communicating persons.

In Example 9, the subject matter of Example 8 may optionally include, wherein the social status of the user and the subject are determined from social media profiles of at least one of the user or subject.

In Example 10, the subject matter of Example 9 may optionally include, wherein the social status includes professional roles between the user and the subject.

In Example 11, the subject matter of any of Examples 1-10 may optionally include, wherein to determine a social-interaction-emotional-state, at least one of the plurality of context attributes is compared to a normalized value corresponding to the at least one of the plurality of context attributes to determine a variance, the variance used to determine the social-interaction-emotional-state from the pre-defined classification methodology.

In Example 12, the subject matter of any of Examples 1-11 may optionally include, wherein the feedback response includes a haptic component.

In Example 13, the subject matter of any of Examples 1-12 may optionally include, wherein the feedback response includes a visual component.

In Example 14, the subject matter of Example 13 may optionally include, wherein the visual component is at least one of a text message, an instant message, a video, or an icon.

In Example 15, the subject matter of Examples 1-14 may optionally include, wherein the feedback response includes an audio component.

In Example 16, the subject matter of any of Examples 1-15 may optionally include, wherein the feedback response includes an environmental component.

In Example 17, the subject matter of any of Examples 1-16 may optionally include a recommendation circuit-set to receive observations of a second interpersonal interaction including attributes of parties to the second interpersonal interaction and a second social-interaction-emotional-state—the attributes including a second plurality of context attributes including a set of interest attributes, assess suitability of the second interpersonal interaction for the user based on both the second social-interaction-emotional-state and the set of interest attributes, and provide the user with a recommendation as to whether to join the second interpersonal interaction based on the assessed suitability of the second interpersonal interaction.

In Example 18, the subject matter of Example 17 may optionally include, wherein the recommendation includes directions to bring the user to the second interpersonal interaction.

In Example 19, the subject matter of any of Examples 1-18 may optionally include a progress adjustment circuit-set to modify a subsequent feedback response based on a deviation of an interaction result for the user from a desired interaction result following receipt of the feedback response by the user—the feedback response and the subsequent feedback response configured to modify a behavior of the user to achieve the desired interaction result for the interpersonal interaction.

Example 20 may include, or may optionally be combined with the subject matter of any of Examples 1-19 to include, subject matter (such as a method, means for performing acts, or machine readable medium including instructions that, when performed by a machine cause the machine to perform acts) comprising receiving—via a transceiver—a plurality of context attributes about a user and a subject, determining—by an interpersonal interaction circuit-set—a period in which an interpersonal interaction between the user and the subject occurs, determining—by an emotional assessment circuit-set—a social-interaction-emotional-state—during the period—from the plurality of context attributes using a pre-defined classification methodology, generating—by a feedback circuit-set—a feedback response based on the determined social-interaction-emotional-state, and presenting the feedback response to the user.

In Example 21, the subject matter of Example 20 may optionally include, wherein the plurality of context attributes includes at least one of an environmental attribute, an emotional attribute, a physical attribute, an interest attribute, an identification attribute, or a social status attribute.

In Example 22, the subject matter of any of Examples 20-21 may optionally include, wherein the plurality of context attributes are derived from sensor data from a sensor of a device, the sensor positioned so as to observe a physical area of the interpersonal interaction.

In Example 23, the subject matter of Example 22 may optionally include, wherein the device is operated by at least one of the user or the subject.

In Example 24, the subject matter of any of Examples 20-23 may optionally include controlling access to a set of at least one device-present context attribute, the set being a subset of the plurality of context attributes, based on an access control list controlled by a provider and identification of a potential recipient, the set having been granted access to the user as the potential recipient.

In Example 25, the subject matter of Example 24 may optionally include, wherein the subject is the provider.

In Example 26, the subject matter of any of Examples 24-25 may optionally include, wherein the access control list includes role based access for a portion of the set, roles in the role based access corresponding to positions in interpersonal communications.

In Example 27, the subject matter of Example 26 may optionally include, wherein the positions in interpersonal communications include a social status of communicating persons.

In Example 28, the subject matter of Example 27 may optionally include, wherein the social status of the user and the subject are determined from social media profiles of at least one of the user or subject.

In Example 29, the subject matter of Example 28 may optionally include, wherein the social status includes professional roles between the user and the subject.

In Example 30, the subject matter of any of Examples 20-29 may optionally include, wherein to determine a social-interaction-emotional-state, at least one of the plurality of context attributes is compared to a normalized value corresponding to the at least one of the plurality of context attributes to determine a variance, the variance used to determine the social-interaction-emotional-state from the pre-defined classification methodology.

In Example 31, the subject matter of any of Examples 20-30 may optionally include, wherein the feedback response includes a haptic component.

In Example 32, the subject matter of any of Examples 20-31 may optionally include, wherein the feedback response includes a visual component.

In Example 33, the subject matter of Example 32 may optionally include, wherein the visual component is at least one of a text message, an instant message, a video, or an icon.

In Example 34, the subject matter of any of Examples 20-33 may optionally include, wherein the feedback response includes an audio component.

In Example 35, the subject matter of any of Examples 20-34 may optionally include, wherein the feedback response includes an environmental component.

In Example 36, the subject matter of any of Examples 20-35 may optionally include, receiving observations of a second interpersonal interaction including attributes of parties to the second interpersonal interaction and a second social-interaction-emotional-state—the attributes including a second plurality of context attributes including a set of interest attributes, assessing suitability of the second interpersonal interaction for the user based on both the second social-interaction-emotional-state and the set of interest attributes, and providing the user with a recommendation as to whether to join the second interpersonal interaction based on the assessed suitability of the second interpersonal interaction.

In Example 37, the subject matter of Example 36 may optionally include, wherein the recommendation includes directions to bring the user to the second interpersonal interaction.

In Example 38, the subject matter of any of Examples 20-37 may optionally include, modifying a subsequent feedback response based on a deviation of an interaction result for the user from a desired interaction result following receipt of the feedback response by the user—the feedback response and the subsequent feedback response configured to modify a behavior of the user to achieve the desired interaction result for the interpersonal interaction.

Example 39 may include, or may optionally be combined with the subject matter of any one of Examples 1-38 to include subject matter (such as a device, apparatus, or system for social cuing based on in-context observation) including at least one machine readable medium including instructions that, when executed by a machine, cause the machine to perform any of Examples 20-38.

Example 40 may include, or may optionally be combined with the subject matter of any one of Examples 1-39 to include subject matter (such as a device, apparatus, or system for social cuing based on in-context observation) including a system comprising means to perform any of Examples 20-38.

Example 41 may include, or may optionally be combined with the subject matter of any one of Examples 1-40 to include, subject matter (such as a device, apparatus, or system for social cuing based on in-context observation) comprising, receipt means for receiving a plurality of context attributes about a user and a subject, timing means for determining a period in which an interpersonal interaction between the user and the subject occurs, emotional state means for determining a social-interaction-emotional-state—during the period—from the plurality of context attributes using a pre-defined classification methodology, response means for generating a feedback response based on the determined social-interaction-emotional-state, and presentation means for presenting the feedback response to the user.

In Example 42, the subject matter of Example 41 may optionally include, wherein the plurality of context attributes includes at least one of an environmental attribute, an emotional attribute, a physical attribute, an interest attribute, an identification attribute, or a social status attribute.

In Example 43, the subject matter of any of Examples 41-42 may optionally include, wherein the plurality of context attributes are derived from sensor data from a sensor of a device, the sensor positioned so as to observe a physical area of the interpersonal interaction.

In Example 44, the subject matter of Example 43 may optionally include, wherein the device is operated by at least one of the user or the subject.

In Example 45, the subject matter of any of Examples 41-44 may optionally include an access control mechanism to control access to a set of at least one device-present context attribute, the set being a subset of the plurality of context attributes, based on an access control list controlled by a provider and identification of a potential recipient, the set having been granted access to the user as the potential recipient.

In Example 46, the subject matter of Example 45 may optionally include, wherein the subject is the provider.

In Example 47, the subject matter of any of Examples 45-46 may optionally include, wherein the access control list includes role based access for a portion of the set, roles in the role based access corresponding to positions in interpersonal communications.

In Example 48, the subject matter of Example 47 may optionally include, wherein the positions in interpersonal communications include a social status of communicating persons.

In Example 49, the subject matter of Example 48 may optionally include, wherein the social status of the user and the subject are determined from social media profiles of at least one of the user or subject.

In Example 50, the subject matter of Example 49 may optionally include, wherein the social status includes professional roles between the user and the subject.

In Example 51, the subject matter of any of Examples 41-50 may optionally include, wherein to determine the social-interaction-emotional-state, at least one of the plurality of context attributes is compared to a normalized value corresponding to the at least one of the plurality of context attributes to determine a variance, the variance used to determine the social-interaction-emotional-state from the pre-defined classification methodology.

In Example 52, the subject matter of any of Examples 41-51 may optionally include, wherein the feedback response includes a haptic component.

In Example 53, the subject matter of any of Examples 41-52 may optionally include, wherein the feedback response includes a visual component.

In Example 54, the subject matter of Example 53 may optionally include, wherein the visual component is at least one of a text message, an instant message, a video, or an icon.

In Example 55, the subject matter of any of Examples 41-54 may optionally include, wherein the feedback response includes an audio component.

In Example 56, the subject matter of any of Examples 41-55 may optionally include, wherein the feedback response includes an environmental component.

In Example 57, the subject matter of any of Examples 41-56 may optionally include remote observation means for receiving observations of a second interpersonal interaction including attributes of parties to the second interpersonal interaction and a second social-interaction-emotional-state—the attributes including a second plurality of context attributes including a set of interest attributes, compatibility means for assessing suitability of the second interpersonal interaction for the user based on both the second social-interaction-emotional-state and the set of interest attributes, and recommendation means for providing the user with a recommendation as to whether to join the second interpersonal interaction based on the assessed suitability of the second interpersonal interaction.

In Example 58, the subject matter of Example 57 may optionally include, wherein the recommendation includes directions to bring the user to the second interpersonal interaction.

In Example 59, the subject matter of any of Examples 41-58 may optionally include training means for modifying a subsequent feedback response based on a deviation of an interaction result for the user from a desired interaction result following receipt of the feedback response by the user—the feedback response and the subsequent feedback response configured to modify a behavior of the user to achieve the desired interaction result for the interpersonal interaction.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A system for social cuing based on in-context observation, the system comprising:
a context circuit-set to receive a plurality of context attributes about a user and a subject;
an interpersonal interaction circuit-set to determine a period in which an interpersonal interaction between the user and the subject occurs;
an emotional assessment circuit-set to determine a social-interaction-emotional-state, during the period, from the plurality of context attributes using a pre-defined classification methodology;
a feedback circuit-set to generate a feedback response based on the determined social-interaction-emotional-state; and
a presentation circuit-set to present the feedback response to the user.

2. The system of claim 1, wherein the plurality of context attributes are derived from sensor data from a sensor of a device, the sensor positioned so as to observe a physical area of the interpersonal interaction.

3. The system of claim 1, including an access control mechanism to control access to a set of at least one device-present context attribute, the set being a subset of the plurality of context attributes, based on an access control list controlled by a provider and identification of a potential recipient, the set having been granted access to the user as the potential recipient.

4. The system of claim 3, wherein the access control list includes role based access for a portion of the set, roles in the role based access corresponding to positions in interpersonal communications.

5. The system of claim 4, wherein the positions in interpersonal communications include a social status of communicating persons.

6. The system of claim 1, wherein to determine a social-interaction-emotional-state, at least one of the plurality of context attributes is compared to a normalized value corresponding to the at least one of the plurality of context attributes to determine a variance, the variance used to determine the social-interaction-emotional-state from the pre-defined classification methodology.

7. The system of claim 1, including a recommendation circuit-set to:
receive observations of a second interpersonal interaction including attributes of parties to the second interpersonal interaction and a second social-interaction-emotional-state, the attributes including a second plurality of context attributes including a set of interest attributes;
assess suitability of the second interpersonal interaction for the user based on both the second social-interaction-emotional-state and the set of interest attributes; and
provide the user with a recommendation as to whether to join the second interpersonal interaction based on the assessed suitability of the second interpersonal interaction.

8. The system of claim 7, wherein the recommendation includes directions to bring the user to the second interpersonal interaction.

9. A method for social cuing based on in-context observation comprising:
receiving, via a transceiver, a plurality of context attributes about a user and a subject;
determining, by an interpersonal interaction circuit-set, a period in which an interpersonal interaction between the user and the subject occurs;
determining, by an emotional assessment circuit-set, a social-interaction-emotional-state, during the period, from the plurality of context attributes using a pre-defined classification methodology;
generating, by a feedback circuit-set, a feedback response based on the determined social-interaction-emotional-state; and
presenting the feedback response to the user.

10. The method of claim 9, wherein the plurality of context attributes are derived from sensor data from a sensor of a device, the sensor positioned so as to observe a physical area of the interpersonal interaction.

11. The method of claim 9, including controlling access to a set of at least one device-present context attribute, the set being a subset of the plurality of context attributes, based on an access control list controlled by a provider and identification of a potential recipient, the set having been granted access to the user as the potential recipient.

12. The method of claim 11, wherein the access control list includes role based access for a portion of the set, roles in the role based access corresponding to positions in interpersonal communications.

13. The method of claim 12, wherein the positions in interpersonal communications include a social status of communicating persons.

14. The method of claim 9, wherein to determine a social-interaction-emotional-state, at least one of the plurality of context attributes is compared to a normalized value corresponding to the at least one of the plurality of context attributes to determine a variance, the variance used to determine the social-interaction-emotional-state from the pre-defined classification methodology.

15. The method of claim 9, including:
receiving observations of a second interpersonal interaction including attributes of parties to the second interpersonal interaction and a second social-interaction-emotional-state, the attributes including a second plurality of context attributes including a set of interest attributes;
assessing suitability of the second interpersonal interaction for the user based on both the second social-interaction-emotional-state and the set of interest attributes; and
providing the user with a recommendation as to whether to join the second interpersonal interaction based on the assessed suitability of the second interpersonal interaction.

16. The method of claim 15, wherein the recommendation includes directions to bring the user to the second interpersonal interaction.

17. At least one machine-readable medium including instructions that, when executed by a machine, cause the machine to perform operations comprising:
receiving, via a transceiver, a plurality of context attributes about a user and a subject;
determining, by an interpersonal interaction circuit-set, a period in which an interpersonal interaction between the user and the subject occurs;
determining, by an emotional assessment circuit-set, a social-interaction-emotional-state, during the period, from the plurality of context attributes using a pre-defined classification methodology;
generating, by a feedback circuit-set, a feedback response based on the determined social-interaction-emotional-state; and
presenting the feedback response to the user.

18. The at least one machine-readable medium of claim 17, wherein the plurality of context attributes are derived from sensor data from a sensor of a device, the sensor positioned so as to observe a physical area of the interpersonal interaction.

19. The at least one machine-readable of claim 17, including controlling access to a set of at least one device-present context attribute, the set being a subset of the plurality of context attributes, based on an access control list controlled by a provider and identification of a potential recipient, the set having been granted access to the user as the potential recipient.

20. The at least one machine-readable medium of claim 19, wherein the access control list includes role based access for a portion of the set, roles in the role based access corresponding to positions in interpersonal communications.

21. The at least one machine-readable medium of claim 20, wherein the positions in interpersonal communications include a social status of communicating persons.

22. The at least one machine-readable medium of claim 17, wherein to determine a social-interaction-emotional-state, at least one of the plurality of context attributes is compared to a normalized value corresponding to the at least one of the plurality of context attributes to determine a variance, the variance used to determine the social-interaction-emotional-state from the pre-defined classification methodology.

23. The at least one machine-readable medium of claim 17, including:
receiving observations of a second interpersonal interaction including attributes of parties to the second interpersonal interaction and a second social-interaction-emotional-state, the attributes including a second plurality of context attributes including a set of interest attributes;
assessing suitability of the second interpersonal interaction for the user based on both the second social-interaction-emotional-state and the set of interest attributes; and
providing the user with a recommendation as to whether to join the second interpersonal interaction based on the assessed suitability of the second interpersonal interaction.

24. The at least one machine-readable medium of claim 23, wherein the recommendation includes directions to bring the user to the second interpersonal interaction.
